(19) United States
(12) Patent Application Publication
(10) Pub. No.: US 2017/0246414 A1
(43) Pub. Date: Aug. 31, 2017

(54) BREATHING MASK WITH NASAL CANNULA

(71) Applicant: KING SAUD UNIVERSITY, RIYADH (SA)

(72) Inventor: AHMED SALEM O. BAHAMMAM, RIYADH (SA)

(21) Appl. No.: 15/054,054

(22) Filed: Feb. 25, 2016

(51) Int. Cl. A61M 16/06; A61M 16/08

(52) U.S. Cl. CPC A61M 16/0672; A61M 16/0875; A61M 16/0683

(57) ABSTRACT

The breathing mask with nasal cannula includes a breathing mask having a frontal air inlet port that communicates with an air inlet in a removable mask retainer. The air inlet of the mask retainer is adapted to receive ambient air provided by a non-invasive ventilation (NIV) device. In addition, the mask includes an oxygen pass-through connector tube that is sealed in place in the front of the mask. A nasal cannula is connected to the interior portion of the connector tube and adhesively secured to the patient's nose. The mask and retainer are then secured over the patient's nose and mouth, and an oxygen supply tube is connected to the external end of the pass-through tube of the mask. Airflow is adjusted from the NIV and to oxygen flow is adjusted to the cannula to provide the patient with the required air and oxygen flow for proper blood saturation.

### BACKGROUND OF THE INVENTION

1. Field of the Invention

The present invention relates generally to auxiliary or supplemental air and/or oxygen delivery devices and systems, and particularly to a breathing mask with a nasal cannula.

2. Description of the Related Art

Supplemental air and/or oxygen delivery to patients is a well-known treatment for a number of illnesses and conditions. There are essentially two treatments that may be applied for patients with respiratory difficulties, which are administered according to the specific respiratory problem of the patient. In cases where the oxygen needs of the patient are not acute, a non-invasive ventilation (NIV) device may provide sufficient oxygen for breathing. NIV devices typically comprise relatively low pressure fans or other means for providing a slight positive pressure of ambient air to the patient via a breathing mask. For a patient requiring additional oxygen, an oxygen supply (e.g., pressurized cylinder, etc.) provides oxygen through a regulator and mask or cannula.

In many cases, patients who use an NIV device require some additional oxygen, at least periodically. When such patients are hospitalized, the specialized ventilators available can be precisely controlled to monitor and meter the inspired oxygen by means of their separate inhalation and exhalation tubes. However, most ventilators available for home and portable use have only an inhalation tube with an exhalation port to vent exhaled carbon dioxide, and do not provide measurement or control of the oxygen fraction. In these cases, patients who require supplemental oxygen in addition to that provided by the ventilator (NIV) generally receive supplemental oxygen that is bled into the mask or the ventilator circuit. It will be seen that this results in imprecise control of the fraction inspired oxygen (FiO₂) delivered to the patient. The FiO₂ delivered to the patient is affected by a number of different factors, e.g., intentional or unintentional leaks or seepage from the mask, pressure settings of the NIV unit, oxygen flow and interface of the flow with the mask, etc.

In many cases supplemental oxygen is connected to a port provided in the mask at some point. The problem with this arrangement is that some indeterminate fraction of the delivered oxygen passes out of the mask through the conventional exhalation port of the mask, thus making it impossible to set a precise FiO₂ level for the patient. In addition, the same potential problems exist with such a ported oxygen delivery mask as with other oxygen delivery systems, i.e., seepage around the rim of the mask, etc. Thus, patients often receive lower oxygen fractions or concentrations, and correspondingly low oxygen saturation in the blood, than desired.

Attempts to avoid the above problems have been made by first placing an oxygen delivery cannula on the patient, and then fitting an NIV mask over the patient's nose and mouth. While this does provide some improvement in precise oxygen delivery and control, there is still some leakage at the rim of the mask due to the oxygen delivery tube to the cannula passing between the rim of the mask and the patient's face. This can also result in some additional discomfort to the patient due to the discontinuity of the seal of the mask to the face of the patient.

Thus, a breathing mask with nasal cannula solving the aforementioned problems is desired.

### SUMMARY OF THE INVENTION

The breathing mask with nasal cannula includes a breathing mask having a removably attached retainer and a removably attached cannula. The mask retainer includes a fitting for a non-invasive ventilation (NIV) hose or tube, the tube having an exhalation port in the top of the tube adjacent the retainer attachment. The mask retainer fitting attaches to a seal in the front of the mask. The mask includes a front port for the cannula supply tube, the front port having a short connecting tube installed therethrough with a seal between the connecting tube and the mask structure. The cannula includes specially configured adhesive pads to each side thereof, to secure the cannula to the sides of the nose of the patient.

Initially, the cannula is adhesively secured to the nose of the patient, the two nostril insertion tubes or oxygen delivery prongs extending into the nostrils of the patient. The oxygen input tube of the cannula is then affixed to the internal end of the pass-through tube of the mask and the mask is secured over the nose and mouth of the patient by means of the mask retainer. The oxygen supply tube is then connected to the external end of the pass-through connection tube through the front of the mask, and oxygen flow is initiated and adjusted as required. The non-invasive ventilation (NIV) device is connected to the airflow inlet of the retainer, which serves as an outlet to the mask, communicating with and being sealed to the airflow inlet of the mask. The NIV device is adjusted to provide the patient with proper ambient airflow and supplementary oxygen through the cannula and its connection through the front of the mask.

These and other features of the present invention will become readily apparent upon further review of the following specification and drawings.

### BRIEF DESCRIPTION OF THE DRAWINGS

FIG. 1 is an exploded perspective view of a breathing mask with nasal cannula according to the present invention, illustrating its general features.

FIG. 2 is an environmental perspective view of the breathing mask with nasal cannula of FIG. 1, illustrating the apparatus in use.

FIG. 3 is an environmental perspective view of a conventional breathing mask and non-invasive ventilation device of the prior art.

FIG. 4 is an environmental perspective view of a conventional breathing mask with supplemental oxygen delivery tube of the prior art.

Similar reference characters denote corresponding features consistently throughout the attached drawings.

### DETAILED DESCRIPTION OF THE PREFERRED EMBODIMENTS

The breathing mask with nasal cannula provides for simultaneous delivery of both air and supplemental pure oxygen to a patient, the cannula delivering oxygen directly into the nasal passages of the patent. This ensures better oxygen saturation of the blood and enables more accurate monitoring of oxygen delivery into the patient's respiratory system.

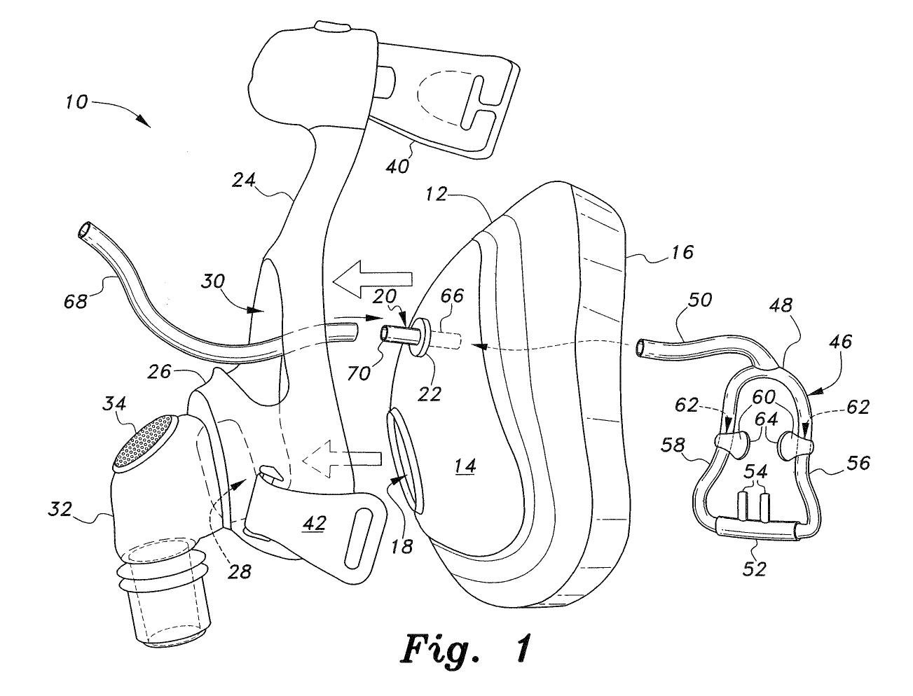

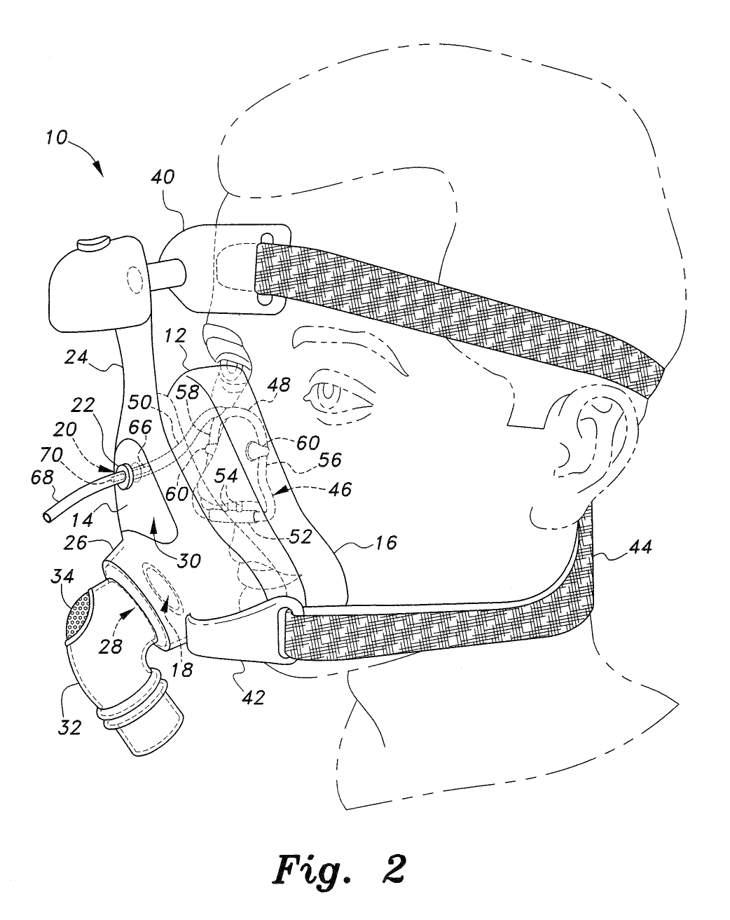

FIGS. 1 and 2 respectively illustrate an exploded perspective view of the breathing mask with nasal cannula apparatus 10, and an environmental perspective view of the apparatus 10 placed upon a patient for use. The apparatus 10 includes a breathing mask 12 having a front portion 14 and a soft, resilient face seal portion or rim 16. The front portion 14 of the mask 12 includes a relatively large diameter air delivery port 18 and a smaller diameter oxygen delivery tube 20 disposed through the front portion 14, the air delivery port 18 and the oxygen delivery tube 20 being separated from one another in the front portion 14 of the mask 12. A seal or grommet 22 is provided about the oxygen delivery tube 20 to provide a substantially gas-tight seal. The mask 12 is otherwise devoid of passages therethrough, as can be seen in FIGS. 1 and 2, in order to preclude leakage of oxygen or air therethrough.

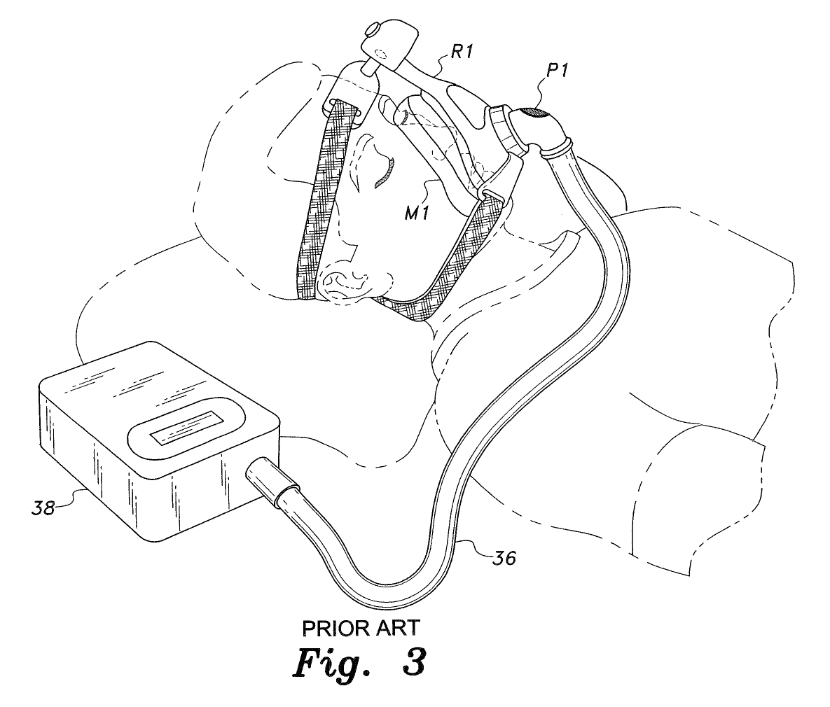

A mask retainer 24 is provided to secure and retain the mask 12 securely and to seal the mask 12 over the nose and mouth of the patient. The retainer 24 includes a front portion 26 having an air tube connection passage 28 disposed therethrough, and a relatively large open passage 30 disposed above the air tube connection passage 28. An air tube delivery fitting 32 is secured to the front portion 26 of the mask retainer 24 through a sealed port to communicate with the air tube connection passage 28. An exhalation port 34 is provided in the air delivery fitting 32. An air supply hose or tube 36 (shown in prior art FIG. 3) may be removably attached to the air tube delivery fitting 32 to deliver air from a non-invasive ventilation (NIV) device 38 (also shown in FIG. 3). The mask retainer 24 further includes upper and lower attachment straps 40 and 42. As shown in FIG. 2, a mask and retainer securing strap arrangement 44 extends from the attachment straps 40 and 42 to hold the retainer 24, and thus the mask 12, securely to the face of the patient.

A nasal cannula 46 is also provided with the apparatus 10 to deliver pure oxygen (and/or other gas/gases, as required) directly to the nasal passages of the patient. It will be seen that the nasal cannula 46 is in the general form of a closed loop, having an upper portion 48 with an oxygen input tube 50 extending therefrom and a generally diametrically opposite lower portion 52 with two nostril insertion tubes or prongs 54 extending therefrom. The tubes or prongs 54 serve as the oxygen output portion of the cannula 46. The loop of the cannula 46 further includes laterally and generally diametrically opposite left and right side tubes 56 and 58, having corresponding nose pads 60 disposed upon each of the two side tubes 56, 58. Each nose pad 60 comprises a cushion formed of a soft, resilient material and has a generally toroidal ring defining an annular passage 62 extending behind the cushion. The passage 62 provides for the insertion of the tube of the cannula 46 therethrough. The mutually facing surfaces of the two pads or cushions 60 are coated with an adhesive surface 64 for securing the cannula 46 temporarily to the nose of the patient.

The breathing mask with nasal cannula apparatus 10 is applied over the nose and mouth of the patient, generally as shown in FIG. 2 of the drawings. Initially the cannula 46 is secured to the nose of the patient by means of the cushions or pads 60 and their adhesive coatings or surfaces 64, and with the two nostril insertion tubes or oxygen delivery prongs 54 extending into the nostrils of the patient. The oxygen input tube 50 of the cannula 46 is then affixed to the internal end 66 of the pass-through tube 20 of the mask 12, and the mask 12 is secured over the nose and mouth of the patient by means of the mask retainer 24. The oxygen supply tube 68 is then connected to the external end 70 of the pass-through connection tube 20 on the front 14 of the mask 12, and oxygen flow is initiated and adjusted as required. The non-invasive ventilation (NIV) device 38 is connected to the airflow inlet 28 of the retainer 24 by means of the air tube delivery fitting 32. The airflow inlet 28 of the retainer 24 serves as an outlet to the mask 12, and communicates with and is sealed to the airflow inlet 18 of the mask 12. The NIV device 38 is adjusted to provide the patient with proper ambient airflow, along with supplemental oxygen provided through the cannula 46 and its connection through the front 14 of the mask 12.

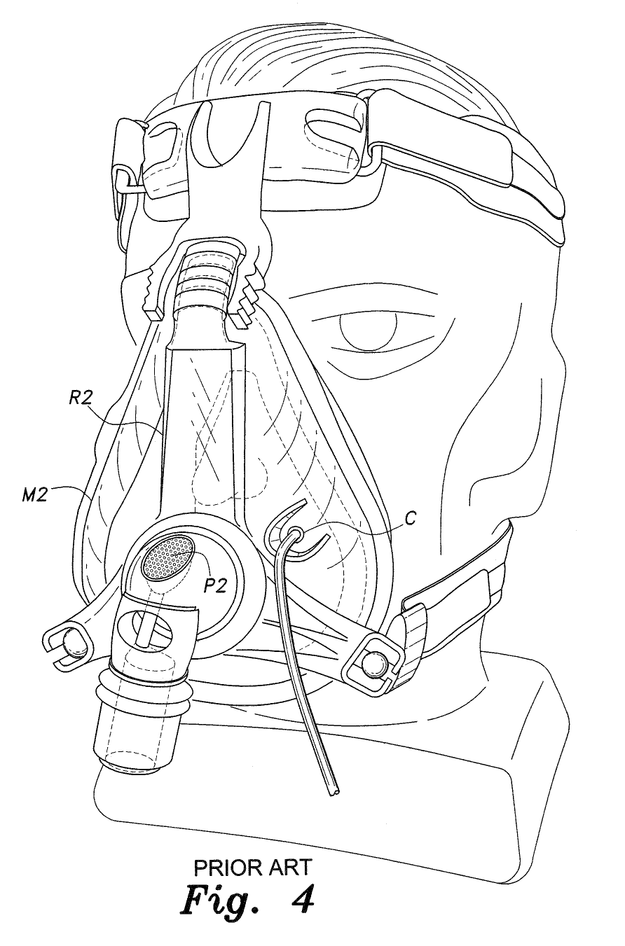

FIGS. 3 and 4 illustrate prior art devices for supplying a patient with additional air and/or oxygen. In FIG. 3, a conventional mask M1 receives air from the NIV device 38 through the delivery hose or tube 36. The mask M1 is devoid of any other passages for receiving oxygen (and/or other gas or gases) from another source. In the prior art apparatus of FIG. 3, if additional pure oxygen is to be administered to the patient, an oxygen supply tube must be run beneath the edge or rim of the mask M1 between the skin of the patient's face and the rim of the mask M1. This results in some leakage at the location of the oxygen supply tube, as well as irritation to the patient's face due to the discontinuity of the supply tube passing under the edge or rim of the mask M1. Moreover, it is impossible to achieve an accurate oxygen fraction and oxygen saturation of the blood when such leakage occurs at the edge of the mask M1.

In FIG. 4, another prior art mask M2 is provided with an oxygen inlet connection C, to which an external oxygen supply line or tube, as in the tube 68 of FIGS. 1 and 2, is connected. This obviates the problem of leakage and discomfort around such a tube being inserted beneath the edge or rim of the mask, but still does not supply pure oxygen directly to the nasal passages of the patient. The same problem exists with the prior art apparatus of FIG. 4 as in the apparatus of FIG. 3, i.e., the administered oxygen flows into the volume between the mask and the face of the patient, so that at least some of the administered oxygen escapes through the exhalation passage P2 without being inhaled by the patient. The present breathing mask and nasal cannula apparatus 10 of FIGS. 1 and 2 solves this problem by providing oxygen flow directly to the nasal passages of the patient, while simultaneously delivering air from the NIV device, without disrupting the seal of the mask over the face of the patient.

It is to be understood that the present invention is not limited to the embodiments described above, but encompasses any and all embodiments within the scope of the following claims.

1. A breathing mask with nasal cannula, comprising:
a breathing mask having a front portion, the front portion including an air delivery port;
an oxygen delivery tube disposed through the front portion of the mask, the oxygen delivery tube being separate from the air delivery port, wherein the oxygen delivery tube extending beyond the front portion of the mask includes oxygen input tube;
a mask retainer, the mask retainer having a front portion, the front portion having an air tube connection passage disposed therethrough and an open passage disposed therethrough above the air tube connection passage, the mask retainer being disposed over the breathing mask and having means for retaining the breathing mask over a patient's face; and
a nasal cannula, the cannula having the oxygen input tube extending therefrom, wherein the nasal cannula consists of a one-piece, continuous closed loop configuration, including a lower portion having two nostril insertion tubes extending therefrom, an upper portion having the oxygen input tube extending therefrom, the oxygen input tube diametrically above and opposite the nostril insertion tubes, and laterally and diametrically opposite left and right side tubes and a nose pad disposed upon each of the side tubes, each of the nose pads being a resilient cushion having mutually facing adhesive surfaces for securing the nasal cannula to the patient's nose.

2. The breathing mask with nasal cannula according to claim 1,
wherein: the breathing mask is otherwise devoid of passages therethrough, other than the air tube connection passage and the oxygen delivery tube.

3. (canceled)

4. The breathing mask with nasal cannula according to claim 1, further comprising:
a sealed fitting extending through the air tube connection passage in said mask retainer and through the air delivery port in said breathing mask, the fitting having an exhalation port disposed therein; and
an air delivery tube attached to the sealed fitting, the air delivery tube being adapted for connection to a non-invasive ventilation (NIV) device.

5. The breathing mask with nasal cannula according to claim 1, wherein said means for retaining comprises upper and lower strap attachments extending from the mask retainer.
